(19) United States
(12) Patent Application Publication
(10) Pub. No.: US 2008/0318327 A1
(43) Pub. Date: Dec. 25, 2008

(54) Kit and Method for Mass Labelling

(75) Inventors: Bengt Bjellqvist, Uppsala (SE); David Fenyo, New York, NY (US); Henrik Neu, Uppsala (SE); Jesper Hedberg, Stockholm (SE)

Correspondence Address: GE HEALTHCARE BIO-SCIENCES CORP.;PATENT DEPARTMENT, 800 CENTENNIAL AVENUE, PISCATAWAY, NJ, 08855, US

(73) Assignee: GE HEALTHCARE BIO-SCIENCES AB, UPPSALA (SE)

(21) Appl. No.: 12/097,266

(22) PCT Filed: Dec. 11, 2006
(86) PCT No.: PCT/SE2006/001407

§ 371 Date: Jun. 13, 2008

(30) Foreign Application Priority Data
Jan. 5, 2006 (SE) 0600032-7

(51) Int. Cl. G01N 33/68

(52) U.S. Cl. 436/89

(57) ABSTRACT

The present invention relates to a kit for mass labelling of peptides in two or more samples, comprising a set of mass tagging reagents for reaction at the N-terminals of the peptides and a set of mass balancing reagents for reaction at the C-terminals of the peptides, or vice versa, and wherein each mass tagging reagent in the set is matched with mass balancing reagent(s) in such a way that the sum of the masses from all matched mass tagging and mass balancing reagents is equal. The invention also relates to a method of using said mass labels and a database for use in said method.

### FIELD OF THE INVENTION

The present invention is within the field of proteomics. More closely, the invention relates to a kit and method for peptide analysis by global mass tagging of peptides.

### BACKGROUND OF THE INVENTION

The peptide based techniques presently used for differential analysis in proteomic studies normally contain the following steps: mass tagging, followed by digestion, ion exchange and/or some type of complexity reduction like ICAT (Isotope Coded Affinity Tags) disclosed in WO 00/11208 or COFRADIC (Combined Fractional Diagonal Chromatographic) method disclosed in WO 02/07716 combined with reversed phase chromatography (RPC) and finally identification and relative quantification with mass spectrometry (MS).

Global tagging aimed at relative quantification of peptides requires a technique independent of amino acid composition or posttranslational modifications, and is normally done after tryptic digestion of the proteins at the N- or C-terminal of the peptides [1,2,3]. Most commonly used is presumably acylation at the N-terminal at neutral pH with N-hydroxysuccinimide (NHS) ester, but at these conditions not only the N-terminal but also the ε-aminogroup of the lysines will react. To avoid the latter reaction, guanidation of the lysine group with O-methylisourea can be done prior to acylation [4]. An advantage of this approach is that this modification result in an increased ionisation efficiency for lysine containing peptides. Besides NHS-esters there exist a large number of other reagents possible to use with primary amino acids for example 2,4 dinitrofluorobenzene [5], phenylisothiocyanate [6] or reaction with aldehyde followed by reduction [7]. Specific global tagging at the C-terminal has so far been done with ¹⁸O. Trypsin, chymotrypsin, lys-C and glu-C introduces up to two ¹⁸O atoms when the proteolysis is done in H₂¹⁸O. This enzymes will also catalyse the inclusion of two ¹⁸O atoms pro peptide in already digested peptide mixture at the positions corresponding to the cleavage sites of the enzyme used [8,9].

The use of internally balanced mass tags in global mass tagging has been described in WO 01/68664 and WO 04/070352. In their approaches, the N-terminal participates in a reaction transferring a group containing a mass tagged low molecular weight reporter group as well as a group contributing to mass balance, where the reporter and mass balance are split apart in the fragmentation step. A limitation with this approach is that peptides with the same retention times and masses are contributing to the background signal, e.g., a mass peak originating from the tail of the isotope distribution of a peak with lower molecular weight or any peptide/protein present in low concentrations, will release the same low molecular reporter molecule. Therefore, the MS/MS signal from the peptide of interest will be indistinguishable from the MS/MS signal contributed from background noise. This will limit the possibility to make relative concentration determinations for low abundant peptides/proteins.

### SUMMARY OF THE INVENTION

The present invention provides means and methods for relative concentration determinations of low abundant peptides/proteins in sample(s). The present invention enables this by providing a new global mass tagging strategy, i.e. one that starts with digestion followed by tagging involving reactions on the N as well as on the C-terminal.

In a first aspect, the invention relates to a kit for mass labelling of peptides in two or more samples, comprising a set of mass tagging reagents for reaction at the N-terminals of the peptides and a set of mass balancing reagents for reaction at the C-terminals of the peptides, or vice versa, and wherein each mass tagging reagent in the mass tagging set is matched, or used together with, mass balancing reagent(s) in such a way that the sum of the masses from all matched mass tagging and mass balancing reagents is equal. The invention relates to a kit for mass labelling of peptides in two or more samples, wherein the amino acid residues at one end, either the N-terminal or C-terminal end, of the peptides present in different samples are reacted with reagents, and as a result of differences in the isotopic composition of the reagents used with different samples the mass increase transferred to the peptides in the reaction(s) differ between the samples; and wherein the amino acid residues at the other end of the peptides are reacted with other reagents with an isotopic composition which for the different samples are chosen so that the sum of the mass increases of the peptides resulting from the reaction of N-terminal amino acid and the reaction of the C-terminal amino acid, at least for a fraction of the peptides, add up to the same value for all samples. Preferably, one of the reagents in the method is H₂¹⁸O, H₂¹⁶O or a mixture of these.

The samples may be complex samples which are enzymatically or chemically digested to generate peptides from proteins. Any endoprotease can be used for this purpose, such as LysC, ArgC, AspN, but preferably trypsin is used. For chemical digestion, for example cyanogens bromide may be used.

Preferably, the kit comprises the following mass tagging reagents: light and heavy forms (D and/or ¹³C forms of) a reagent comprising N-acetoxysuccinimide, N-propoxysuccinimide, acetic anhydride, propionic anhydride, 2,4 dinitrofluorobenzene, phenylisothiocyanate; and the following mass balancing reagents: H₂¹⁸O and optionally H₂¹⁶O.

Alternatively the kit may comprise H¹³CO, H¹²CO, NaBH₄, NaBD₄ and H₂¹⁸O.

In a second aspect, the invention relates to a method for peptide analysis of two or more samples using a kit for mass labelling comprising mass tagging reagents and mass balancing reagents, comprising the following steps:

a) reacting the amino acid residues at one end, either the N-terminal or C-terminal end, of the peptides present in different samples with reagents, and as a result of differences in the isotopic composition of the reagents used with different samples the mass increase transferred to the peptides in the reaction(s) differ between the samples;
b) reacting the amino acid residues at the other end of the peptides, not reacted in step a), with reagents with an isotopic composition which for the different samples are chosen so that the sum of the mass increases of the peptides resulting from the reaction of N-terminal amino acid and the reaction of the C-terminal amino acid, at least for a fraction of the peptides, add up to the same value for all samples; and
c) mass spectrometry analysis of said peptides.

Basically the peptides in the different samples to be used in experiments are modified in two different reactions wherein both reactions involve reagents with isotopic composition which varies with the sample used. The first reaction, beneath described as the tagging reaction, results in a modification at one end of the peptide, either the N-terminal amino acid or the C-terminal amino acid and this modification result in an increase in the peptide mass. As a result of differences in the isotopic composition of the reagent used with the different samples the mass increase transferred to the peptides in this first reaction will differ between the samples. The second reaction beneath described as the mass balancing reaction will involve the other end of the peptide. If the original tagging reaction was done at the N-terminal the mass balancing will be done at the C-termina or vice-versa. The mass increase resulting from the mass balancing will again differ for the peptides between samples as a result of differences in the isotopic composition of the reagents used, but in the mass balancing reactions the isotopic composition of the reagent used with the different samples are chosen so that the sum of the mass increases of the peptides resulting from the tagging reaction and the mass balancing reaction add up to the same value for all samples. After the tagging and mass balancing the samples are mixed. For complex samples some type of high resolution separation is done on the peptides in the resulting mixture and all fractions or selected fractions from said separation are subjected to a separation, such as a chromatographic separation, followed by mass spectrometry. As the sum of the masses added to the peptides is the same in all samples, a peptide originating from different samples will always appear at the same M/z value in the primary spectra. Subjecting ions with a certain M/z-value to fragmentation will result in an MS/MS spectra containing doublets, triplets or quadruplets depending on the numbers of samples used and mixed after the tagging and balancing reactions, and the mass differences within the doublets, triplets or quadruplets reflects the mass differences transferred in the mass tagging and mass balancing reactions. The intensities of the peaks constituting the doublets, triplets or quadruplets allow relative concentration to be determined in the MS/MS mode.

In the method, preferably the N-terminals of peptides in said two or more samples are tagged with heavy forms (D and/or ¹³C forms) or light forms of a reagent comprising N-acetoxysuccinimide, N-propoxysuccinimide, acetic anhydride, propionic anhydride, 2,4 dinitrofluorobenzene, phenylisothiocyanate; and preferably the C-terminals of said at least two samples are mass balanced with a reagent comprising H₂O or H₂¹⁸O; or vice versa, i.e. the N-terminals are mass balanced with the above reagents and the C-terminals are mass tagged with the above reagents.

For differential analysis, use of a light form in combination with a heavy alternative in the mass label is the normal choice, but under certain circumstances other solutions might be required. One example of this is when a mass difference of 4 is created at the C-terminal with the aid of incorporation of two ¹⁸O atoms in one sample and two ¹⁶O atoms in the other and need to be compensated at the N-terminal, while the charge of the N-terminal amino group should be maintained. Charged groups on N-terminal as has an influence on the isoelectric points of peptides and can of this reason be favourable, when focusing is used as a separation method. An alternative in this situation is to react the N-terminal of the sample, already reacted or later to be reacted with ¹⁸O, with ¹³C containing formaldehyde followed by reduction by a hydride to generate a tertiary amino group at the C-terminal with the formula

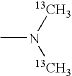

The other sample already reacted or to be reacted with ¹⁶O, with unlabelled formaldehyde followed by reduction with deuteride to generate a tertiary amino group at the C-terminal with the formula

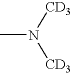

The resulting mass difference at the N-terminal between these two sample is then 4 mass units or identical to the mass difference between two ¹⁸O and two ¹⁶O atoms at the C-terminal.

In the above described embodiment of the method the samples are mass tagged and mass balanced with H¹³CO, H¹²CO, NaBH₄, NaBD₄ and H₂¹⁸O.

In a preferred method for differential analysis of two samples the invention comprises the following steps:
a) preparation of sample 1 and sample 2,
b) digestion of proteins to generate peptides,
c) tagging of N-terminals of peptides by reacting the N-terminals of peptides in sample 1 with a tag with the mass M_{N} and the peptides in sample 2 with a tag with the mass (M_{N}+M_{add}) and mass balancing at the C-terminals thereof by reacting the C-terminals of the peptides of sample 1 with a reactant increasing the mass with (M_{C}+M_{add}) and the peptides of sample 2 with a tag increasing the mass with M_{C} or vice versa;
d) mixing sample 1 and sample 2,
e) performing a high resolution separation of the peptides in the resulting mixture, and
f) subjecting either all fractions or selected fractions resulting from said separation to a chromatographic (preferably reversed phase) separation followed by mass spectrometry where a relative quantification is done in the MS/MS spectra, relating the concentrations of certain peptides in sample 1 to the concentration of the corresponding peptides in sample 2.

In contrast to prior art, global mass tagging according to the present invention is done through a reaction at the N-terminal and mass balance is created through a reaction at the C-terminal, or vice versa. Relative concentrations will be determined from the fragment ions in the MS/MS spectrum. However, these fragment ions will differ from the fragment ions generated from other peptides appearing at the same mass in the primary MS. Provided that the mass of a peptide of interest is known, as well as the masses of the fragment ions resulting from this peptide, it will be possible to collect ions with a mass/charge ratio (M_{peptide}+M_{N}+M_{C}+M_{add})/Z also in the cases when no mass peak is detectable in the primary spectrum. In the resulting MS/MS spectrum the relative concentrations of the peptide in sample 1 and 2, respectively, can be determined from the relative intensities of the peaks appearing as doublets with a difference in mass with M_{add}/Z mass-units at positions known to correspond to the masses of the fragment ions generated from the peptide of interest.

In a third aspect, the invention relates to a database comprising information about the origin and composition of the peptides as well as isoelectric point, retention time in RPC, peptide mass and the masses of the fragments ions appearing in the MS/MS spectrum. The database is preferably arranged in accordance with the method of collecting information about pI, retention time and MS data as described above.

This database provides a novel way to select target protein sub-sets for proteome analysis by MS, for example, protein sub-sets constituting signalling pathways. The tryptic databases correspond to the characterised peptides originating from proteins present in complex samples like human sera, liver or brain. The database information should contain peptide composition including PTMs, identity of the corresponding gene and gene ontology assignments, but also an address to the peptide in a four dimensional analytical space given by the isoelectric point, the retention time in RPC, the peptide mass and the masses of fragment ions in the MS/MS spectrum.

This database will also maximize the dynamic peptide range possible to cover. The data in the database should be generated with peptides tagged at both the N and C-terminal with the reagents transferring the masses M_{N} and M_{C} to the terminals. In the generation of the data base it is clearly advantageous to use the highest resolution feasible in the steps preceding MS.

For differential analysis, another preferred method comprises the following steps:
a) preparation of sample 1 and 2,
b) digestion of proteins to generate peptides,
c) tagging of N-terminals of peptides by reacting the N-terminals of peptides in sample 1 with a tag with the mass M_{N} and the peptides in sample 2 with a tag with the mass (M_{N}+M_{add}),
d) mixing of sample 1 and 2,
e) performing a high resolution separation of the peptides in the resulting mixture,
f) subjecting, either all fractions, or selected fractions resulting in said separation to a chromatographic (preferably reversed phase) separation,
g) after the mixing in step d) but prior to the relative quantification, the C-terminals of the peptides in the mixed sample are reacted with a mixture containing two isotopic variants of the reactant transferring the mass M_{C} or the mass (M_{C}+M_{add}) to the C-terminal, and
h) quantification is done in the MS/MS spectra, relating the concentrations of a certain peptide in sample 1 to the concentration of the corresponding peptide in sample 2 by again collecting ions in the primary spectra with a mass/charge ratio (M_{peptide}+M_{N}+M_{C}+M_{add})/Z.

With the peptide to be analysed initially having a mass equal to M_{peptide} and if, in order to simplify the situation, it is assumed that this peptide is present in equal amounts in sample 1 and sample 2, the result after mixing of samples will be a mixture containing equal amounts of the peptide with the masses (M_{peptide}+M_{N}) and (M_{peptide}+M_{N}+M_{add}) respectively. This mixture is then reacted with a reactant mixture containing two isotopic variants of a reactant transferring to the C-terminal of the peptides the masses M_{C} and (M_{C}+M_{add}), respectively. If, again of simplicity reasons, it is assumed that the reactant mixture contain equal amounts of the isotopic variants of the reactant, the peptide of interest will, in the finally resulting mixture be present with three different masses: (M_{N}+M_{C}) representing 25% of the peptide, (M_{N}+M_{C}+M_{add}) representing 50% of the peptide and (M_{N}+M_{C}+2M_{add}) representing 25% of the peptide. The peak corresponding to the mass (M_{peptide}+M_{N}+M_{add}) selected for generation of a MS/MS spectrum will with the assumptions made contain equal amounts of peptide with the additional mass bound to the N-terminal and C-terminal group, respectively. In the resulting MS/MS spectra the mass peaks relating to the generated fragments will appear as doublets differing in mass with M_{add} mass units. When the value M_{add} is small (1-5 mass units) the peak selected in the primary spectrum will not only contain peptides with the mass generated by adding the mass M_{add} in the reaction with either the N- or C-terminal, but also peptides with only the masses M_{N} and M_{C} added in the reaction and the mass M_{add} contributed by heavy isotopes (mainly ¹³C and ³⁴S) originally present in the peptide. This will cause the peak ratio in the doublets in the MS/MS spectrum to slightly deviate from a 1:1 ratio in the case, when the two samples contain identical amounts of the peptide of interest and the reactant mixture contain equal amounts of the isotopic variants of the reactant used for mass balancing. With the identity of the peptide known and the composition of the fragment corresponding to a peak doublet known, it is from a determination of the ratio between the two peaks in the doublet easy and straight forward to relate the concentration of the peptide in sample 1 to the peptide concentration in sample 2 provided that the ratio of the isotopic variants used in the mass balancing step is known.

In the approach described above the tagging in step c) could alternatively be done at the C-terminal and the reaction with the mixture of isotopic variants in step g) could be done at the N-terminal.

For differential analysis, a further preferred method of the invention comprises the following steps:
a) preparation of sample 1 and 2,
b) digestion of proteins to generate peptides,
c) tagging of N-terminals of peptides by reacting the N-terminals of peptides in sample 1 with a tag with the mass M_{N} and the peptides in sample 2 with a tag with the mass (M_{N}+2),
d) mixing of sample 1 and 2,
e) performing a high resolution separation of the peptides in the resulting mixture,
f) subjecting, either all fractions, or selected fractions resulting in said separation to a chromatographic (preferably reversed phase) separation, and
g) after the mixing in step d) but prior to the relative quantification, addition of H₂¹⁸O together with an enzyme catalysing oxygen exchange between water and the C-terminal carboxyl oxygens. (As the peptides after the H₂¹⁸O addition are solubilised in a H₂¹⁸O/H₂¹⁶O mixture the result is that 0, 1 or 2 are transferred to the C-terminal of the peptides in the mixture.) and
h) quantification is done in the MS/MS spectra, relating the concentrations of a certain peptide in sample 1 to the concentration of the corresponding peptide in sample 2 by selecting in the primary spectra a mass-peak containing peptides reacted to give a mass increase of (M_{N}+2) and/or (M_{N}+4).

Also in this approach the tagging in step c) could be done at the C-terminal and the reaction with a mixture of isotopic variants in step g) could be done at the N-terminal.

In a preferred embodiment, the kit according to the invention comprises N-acetoxysuccinimide+(¹³Cₙ and/or Dₙ) N-acetoxysuccinimide+H₂¹⁸O, wherein n=2 or 4.

Alternatively, the mass label set comprises N-propoxysuccinimide+(¹³Cₙ and/or Dₙ) N-propoxysuccinimide+H₂¹⁸O, wherein n=2 or 4.

In another preferred embodiment, the mass label set comprises acetic anhydride+(¹³Cₙ and/or Dₙ) acetic anhydride+H₂¹⁸O, wherein n=2 or 4.

The mass label set may also comprise propionic anhydride+(¹³Cₙ and/or Dₙ) propionic anhydride+H₂¹⁸O, wherein n=2 or 4.

In a further preferred embodiment, the mass label set comprises formaldehyde+¹³C and/or D formaldehyde+H₂¹⁸O, wherein n=2 or 4. Any other aldehyde may also be used. Furthermore, light and heavy forms of dinitrofluorobenzene and phenylisothiocyanate may also be used [5][6] together with H₂¹⁸O.

The kit with mass tagging and mass balancing reagents may also comprise H₂¹⁶O or mixtures of H₂¹⁸O and H₂¹⁶O.

An advantage of the global tagging according to the invention is that it provides increased possibilities to make measurements on peptides close to the N- and C-terminals to control if an observed concentration difference relates to the full-length protein. Similarly there will be increased possibilities to check the importance of posttranslational modifications (PTMs) or alternative splicing at the site of interest.

A further advantage of global mass tagging according to the invention is that it can accept some incomplete digestion as well as some peptides resulting from chymotryptic activity.

### DETAILED DESCRIPTION OF THE INVENTION

In the method of the invention several different reagents may be used for mass tagging at the N-terminal. Examples of useful mass tagging reagents are:

N-acetoxysuccinimide, N-propoxysuccinimide, propionic anhydride, formaldehyde, or other aldehydes, for generation of dimethyl derivative by reductive amination.

For differential tagging the light reagents contain the normal isotopes and the heavy reagents are substituted with deuterium (Dₙ) or are alternatively substituted with ¹³Cₙ, wherein n is a number from 1-4 depending on the chosen reagent.

One way to balance the N-terminal mass differences caused by the mass tags is to use trypsin to include ¹⁶O and ¹⁸O at the C-terminal either in connection with the tryptic digestion [8] or at a later stage where then trypsin is included together with mass tagged sample peptides in a 1/1 mixture of H₂¹⁶O/H₂¹⁸O to catalyse the ¹⁶O/¹⁸O exchange.

Other ways to balance the N-terminal mass differences are reactions at C-terminal lysines or conversion of lysine to homoarginine followed by the use of reactants with specificity for arginine/homoarginine. This is useful when trypsin is used for digestion of the proteins.

As mentioned above, the mass tagging may also be at the C-terminal in which case the mass balancing is at the N-terminal.

Databases of tryptic peptides with peptide identities and positions in a four dimensional space given by pI, retention time, peptide mass and fragment mass in the MS/MS spectrum will allow standardised methods to be used, not only for concentration determinations, but also for localisation of alternative splicing sites or PTM:s related to disease. In a longer perspective this type of peptide database containing all the information required for the characterisation and concentration determination of the peptides can be seen as a first step towards analytical methods useful in personalised medicine.

### Experimental Part

The invention will now be described in association with some non-limiting examples.

Beneath mass tagging is described at the N-terminal preferably with the aid of NHS ester, transferring a N-terminal mass tagging reagent, see above, containing either none (for light reagent) or two deuterium atoms (for heavy reagent). Balancing the mass of the tagged and untagged peptides is not done in connection with the digestion, but catalytically prior to RPC and MS with the aid of trypsin in water containing ¹⁶O/¹⁸O in the ratio 1/1. For a peptide present in equal amounts in sample and reference a 1:3:3:1 intensity distribution will result for the peaks with the masses M, M+2, M+4 and M+6, respectively.

### 1. Generation of Peptide Data

If a relevant database is not already available, the first step will be to generate the data required for the peptide database covering the samples to be used in later differential display experiments. For these experiments a reference sample is used which might be a mixture of samples covering different condition of biological relevance. The following experimental steps are involved:
1. Sample preparation (solubilisation, denaturation, reduction, protection of cysteinyl residues with DeStreak™ or alkylating agent. Conversion of lysines to homoarginine [8]).
2. Trypsin digestion.
3. Reaction with the NHS-ester later to be used for differential display.
4. Separation of the peptides in complex samples in an isoelectric focusing (IEF) procedure.
5. Identification of peptides in the different IEF-fractions with RPC followed by MS/MS.
6. Compile the accumulated information relating to the peptides including pI, retention time, peptide mass and masses of fragment ions in the MS/MS spectra in a database.

### Peptide Database Covering a Human Proteome

The human genome corresponds to 30-40.000 expressed genes. Tryptic digestion of the products resulting from one gene is expected to give on the average 40 peptides in a M_{w} range suitable for MS detection (mean Mw of a protein of 50 kDa gives 25-30 peptides, alternative splicing and PTM:s adding additionally 10-15 peptides, or totally the genome corresponds to 1.2-1.6 million peptides). In a complex tissue sample it is conceivable that as much as 75% of these genes are expressed.

To generate a database, the prerequisite is that these peptides can be separated in fractions suitably sized for use in MS preceded by RPC and that only a limited number of the peptides are present in more than one of the resulting fractions.

The peptides should be characterised by the identity of the corresponding gene, their composition including PTM:s, the gene ontology assignments (GO) valid for the corresponding gene products and their position as mass tagged peptides in a four dimensional analytical space given by their pI-values, retention times, peptide masses and the fragment masses in the MS/MS spectra. For information on gene ontology assignments see [http://www.ebi.ac.uk/GOA/HUMAN_release.html].

### 2. Differential Display

For differential display experiments, samples and reference are in the three first steps treated in parallel but separately: The following experimental steps are involved.

1. Solubilisation, denaturation and reduction of samples. Protection of cystenyl residues with DeStreak™ or alkylating agent. Convert lysine to homoarginine.
2. Trypsin digestion of samples as well as reference.
3. Reaction of samples with NHS-ester containing no D (light reagent), reaction of reference with NHS-ester containing two D (heavy reagent) atoms transferred to the peptide in the reaction.
4. Mixing of samples with reference.
5. High resolution IEF.
6. Selection of samples for initial use with RPC and MS. Catalytic inclusion of ¹⁶O/¹⁸O in the ratio 1/1 into selected samples. Generations of list of peptides to be compared in differential display. Programming of mass spectrometer instrument with retention times and masses for the peptides to be compared. Peak corresponding to the mass M+2 and/or M+4 to be selected for generation of MS/MS spectra.
7. Evaluation of first set of runs with differential display. Selection of new set of samples to run. Generation of new list of peptides and so on, until the desired information has been collected.

The tagging and mass balancing approach according to the invention is possible to realize with several well described specific reactions. It can be expected that the tagging will give high specificity and very low background noise. The most important advantage of the present invention is that it will allow the coverage of a wide dynamic range of peptides.

### REFERENCES

1. Hsu J-L, Huang S-Y, Chow N-H and Chen S-H, Stable-isotope Dimethyl labeling for quantitative Proteomics Anal. Chem 2002, 75, 6843-6852
2. Liu P and Regnier F E, An Isotope coded strategy for Proteomics Involving Both Amine and Carboxyl Group labelling. J. Proteome Res. 2002, 1, 443-450
3. Chakraborty A and Regnier F E, Global internal standard technology for comparative proteomics J Chromatogr A 2002, 949, 173-184
4. Chen, X. H.; Chen, Y. H.; Anderson, V. E., Protein cross-links: universal isolation and characterization by isotopic derivatization and electrospray ionization mass spectrometry, Anal. Biochem. 1999, 273, 192-203.
5. Zhang, X.; Jin, Q. K.; Carr, S. A. Annan, R. S., N-terminal peptide labeling strategy for incorporation of isotopic tags: a method for the determination of site-specific absolute phosphorylation stoichiometry, Rapid. Commun. Mass Spectrom. 2002, 16, 2325-2332.
6. Mason, D. E.; Liebler, D. C., Quantitative analysis of modified proteins by LC-MS/MS of peptides labeled with phenyl isocyanate, J. Proteome. Res. 2003, 2, 265-272.
7. Zhang, H.; Bart, B. M.; Eng, J.; Aebersold, R. In Mass Spectrometry and Allied Topics, 2003.
8. Schnolzer M, Jedrzejewski P and Lehmann W D,
   Protease-catalyzed incorporation of 18O into peptide fragments and its application for protein sequencing by electrospray and matrix-assisted laser desorption/ionization mass spectrometry. Electrophoresis. 1996 May; 17(5):945-53
9. Yao, X.; Afonso, C.; Fenselau, C. J., Dissection of proteolytic ¹⁸O labeling: endoprotease-catalyzed ¹⁶O-to-¹⁸O exchange of truncated peptide substrates, Proteome Res. 2003, 2, 147-152.

1: A kit for mass labelling of peptides in two or more samples, comprising a set of mass tagging reagents for reaction at the N-terminals of the peptides and a set of mass balancing reagents for reaction at the C-terminals of the peptides, or vice versa, and wherein each mass tagging reagent in the set is matched with mass balancing reagent(s) in such a way that the sum of the masses from all matched mass tagging and mass balancing reagents is equal.

2. The kit of claim 1, comprising the following mass tagging reagents: light and heavy forms (D and/or ¹³C forms of) a reagent comprising N-acetoxysuccinimide, N-propoxysuccinimide, acetic anhydride, propionic anhydride, 2,4 dinitrofluorobenzene, phenylisothiocyanate; and the following mass balancing reagents: H₂¹⁸O and optionally H₂¹⁶O or mixtures thereof.

3. The kit of claim 1, comprising H¹³CO, H¹²CO, NaBH₄, NaBD₄ and H₂¹⁸O.

4. A method for peptide analysis of two or more samples using a kit for mass labelling comprising mass tagging reagents and mass balancing reagents, comprising the following steps:
a) reacting the amino acid residues at one end, either the N-terminal or C-terminal end, of the peptides present in different samples with reagents, and as a result of differences in the isotopic composition of the reagents used with different samples the mass increase transferred to the peptides in the reaction(s) differ between the samples;
b) reacting the amino acid residues at the other end of the peptides with reagents with an isotopic composition which for the different samples are chosen so that the sum of the mass increases of the peptides resulting from the reaction of N-terminal amino acid and the reaction of the C-terminal amino acid, at least for a fraction of the peptides, add up to the same value for all samples; and
c) mass spectrometry analysis of said peptides.

5. The method of claim 4, wherein the N-terminals of peptides in said two or more samples are tagged with heavy forms (D and/or ¹³C forms) or light forms of a reagent comprising N-acetoxysuccinimide, N-propoxysuccinimide, acetic anhydride, propionic anhydride, 2,4 dinitrofluorobenzene, phenylisothiocyanate; and wherein the C-terminals of said at least two samples are mass balanced with a reagent comprising H₂O or H₂¹⁸O; or vice versa.

6. The method of claim 4, wherein the samples are mass tagged and mass balanced with H¹³CO, H¹²CO, NaBH₄, NaBD₄ and H₂¹⁸O.

7. The method of claim 4, comprising a further step d) collecting information about pI, retention time in RPC, peptide mass in MS and fragment ion mass in MS/MS for each peptide or sub-sets of peptides within a database.

8. The method of claim 4, comprising a further step d) comparing pI, retention time in RPC, peptide mass in MS and fragment ion mass in MS/MS for each peptide or sub-sets of peptides with information in pre-established databases comprising information about pI, retention time in RPC, peptide mass in MS and fragment ion mass in MS/MS for peptides of a proteome, or sub-set thereof.

9. A database arranged in accordance with claim 7.

10. The database of claim 9, comprising information about the origin and composition of the peptides as well as isoelectric point, retention time in RPC, peptide mass and the masses of the fragments ions appearing in the MS/MS spectrum.
